(19) United States
(12) Patent Application Publication
(10) Pub. No.: US 2007/0299441 A1
(43) Pub. Date: Dec. 27, 2007

(54) Adjustable Occipital Plate

(75) Inventors: Zachary M. Hoffman, Minneapolis, MN (US); Mark W. Rice, Minneapolis, MN (US); Jason Piehl, Apple Valley, MN (US)

Correspondence Address: WOOD, HERRON & EVANS (ZIMMER SPINE), 2700 CAREW TOWER, 441 VINE STREET, CINCINNATI, OH, 45202, US

(73) Assignees: Zachary M. Hoffman, Minnaepolis, MN (US); Mark W. Rice, Minneapolis, MN (US); Jason Piehl, Apple Valley, MN (US)

(21) Appl. No.: 11/423,201

(22) Filed: Jun. 9, 2006

(51) Int. Cl. A61F 2/30

(52) U.S. Cl. 606/61

(57) ABSTRACT

An occipital plate for use in an occipito-cervico-thoracic (OCT) construct is mounted to a patient's occipital bone. The occipital plate includes an elongate base plate oriented with a midline of the base plate longitudinal on the occipital bone. A pair of lateral sections project in opposite directions from the central section and include attachment assemblies for securing a rod to the occipital plate. The position of the attachment assembly relative to the midline of the occipital plate is adjustable to thereby provide for medial-lateral adjustability. In specific embodiments, the attachment assemblies include a slide member which mates with the lateral section of the occipital plate in a dove-tail joint configuration and a set screw is threadably engaged with the slide member to secure the slide member relative to the midline of the occipital plate and the appropriate spacing and geometry for the specific patient. The set screw also secures a cam, which fixes the rod to the occipital plate.

### TECHNICAL FIELD

This invention relates generally to fixation devices used in orthopedic surgery. This invention relates more particularly to devices used for cervical posterior fixation utilizing a plate attached to a bone in the occipital region and secured to a rod which attaches to a cable, wire, plate, or screw fastened in the spinal region

### BACKGROUND

Fixation devices are used in orthopedic surgery to stabilize bones such as those in the spinal column. One type of fixation device includes a plate attachable to a portion of a bone. The plate may be connected to another bone or another portion of the same bone, directly or through other connecting devices. For example, posterior fixation devices can include a plate fastened to the skull, or occiput, one or more rods running longitudinally along the spine and connected to the plate, and vertebral mounts such as plates, cables, wires, hooks, screws, or other connectors attached to a vertebra and connected to the rod.

A number of such mechanisms are known in the art. To accommodate the variation in patient size and anatomy, a plate often needs to be chosen from a set of plates of multiple sizes and/or varying geometry. This results in a higher cost of the assembly and a need to maintain separate inventory of the various size and geometry occiput plates. It also increases the surgical time because the surgeon must search for the device that best fits the patient. Accordingly, there is a need for an improved fixation plate that reduces inventory and surgery duration while still providing a secure and reliable connection between the rods and the occipital or other bone structure.

### SUMMARY OF THE INVENTION

This invention addresses these and other shortcomings in the prior art. In one embodiment, the invention is directed to a fixation system for connecting a stabilization system to a bone. More specifically, the invention in one embodiment is directed to an occipital plate for use in an occipito-cervico-thoracic (OCT) construct in which an occipital plate is mounted to a patient's occipital bone. Typically, a pair of rods are releasably secured in a spaced relation to each other to the occipital plate and a number of vertebral mounts which may include plates, cables, wires, hooks, screws or other connectors secure the rods relative to specific vertebrae. In one embodiment, the occipital plate includes a base plate with an elongate central section oriented with a midline of the base plate longitudinal on the occipital bone. A pair of lateral sections projects in opposite directions from the central section and include attachment assemblies for securing one of the rods to the occipital plate. The position of the attachment assembly relative to the midline of the occipital plate is adjustable to thereby provide for medial-lateral adjustability when attaching rods to the occipital plate as part of the OCT construct. In specific embodiments, the attachment assemblies include a slide member which mates with the lateral section of the occipital plate in a dove-tail joint configuration. A set screw is threadably engaged with the slide member to secure the slide member relative to the midline of the occipital plate and the appropriate spacing and geometry for the specific patient. The set screw also secures a cam, which fixes the rod to the occipital plate.

This invention also includes a method of attaching a stabilization device such as an occipital plate and an OCT construct to a patient and adjusting the position of the rod relative to the midline of the occipital plate thereby providing media lateral adjustability when attaching the rods.

Therefore, as a result of this invention, a single occipital plate can be utilized for a variety of patient sizes and configurations and provides efficient and secure adjustability in a medial lateral direction when attaching the rods as part of an OCT construct without the need for a large inventory of occipital plates and structures.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a posterior view of an exemplary occipitio-cervico-thoracic construct utilizing an adjustable occipital plate according to one embodiment of this invention;

FIG. 2A is an exploded plan view of the components of one embodiment of the occipital plate according to this invention;

FIG. 2B is a view similar to FIG. 2A with the components assembled together;

FIG. 3 is a cross-sectional view taken along line 3-3 of FIG. 2B; and

FIG. 4 is an elevational view of a rod mounted to the occipital plate according to one embodiment of this invention.

### DETAILED DESCRIPTION OF THE INVENTION

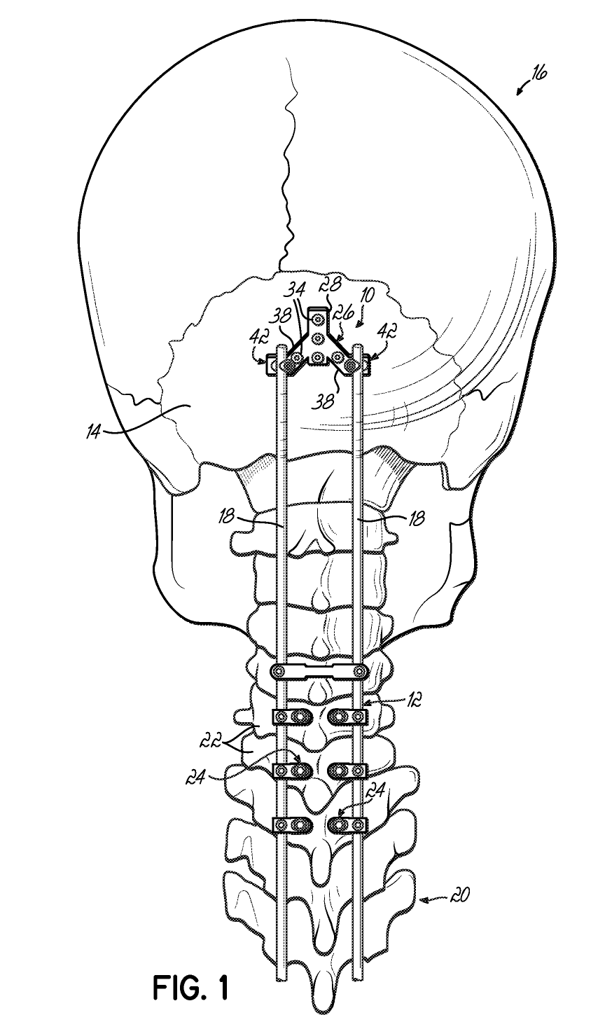

Referring to FIG. 1, one embodiment of an adjustable occipital plate 10 according to this invention is shown utilized as part of an occipito-cervico-thoracic (OCT) construct 12. The adjustable occipital plate 10 in FIG. 1 is mounted to the occipital bone 14 of a patient 16. One or more rods 18 are attached to the occipital plate 10 and run substantially along a spine 20 of the patient 16 and are attached to various segments of the spine such as selected vertebrae 22 by vertebral mounts 24 according to any one of a known variety of such devices.

The occipital plate 10 according to this embodiment of the invention includes a base plate 26 having an elongate central section 28 with a midline 30, which is generally aligned with the spine when mounted to the occipital bone 14. The central section 28 may include a number of holes or apertures 32, three of which are shown, for bone screws 34 to mount the occipital plate 10 to the occipital bone 14 or another portion of the patient 16. The occipital plate 10 is generally symmetric relative to the midline 30 and includes a pair of lateral sections 36 projecting laterally outward in opposite directions from the midline 30. Each lateral section 36 is joined to the central section 28 via an angled transition section 38 that may include a hole or aperture 40 to receive a bone screw 34 there through for an additional mounting location to the occipital or other bone. The lateral sections 36 extend laterally in a direction generally perpendicular to the midline 30 of the central section 28.

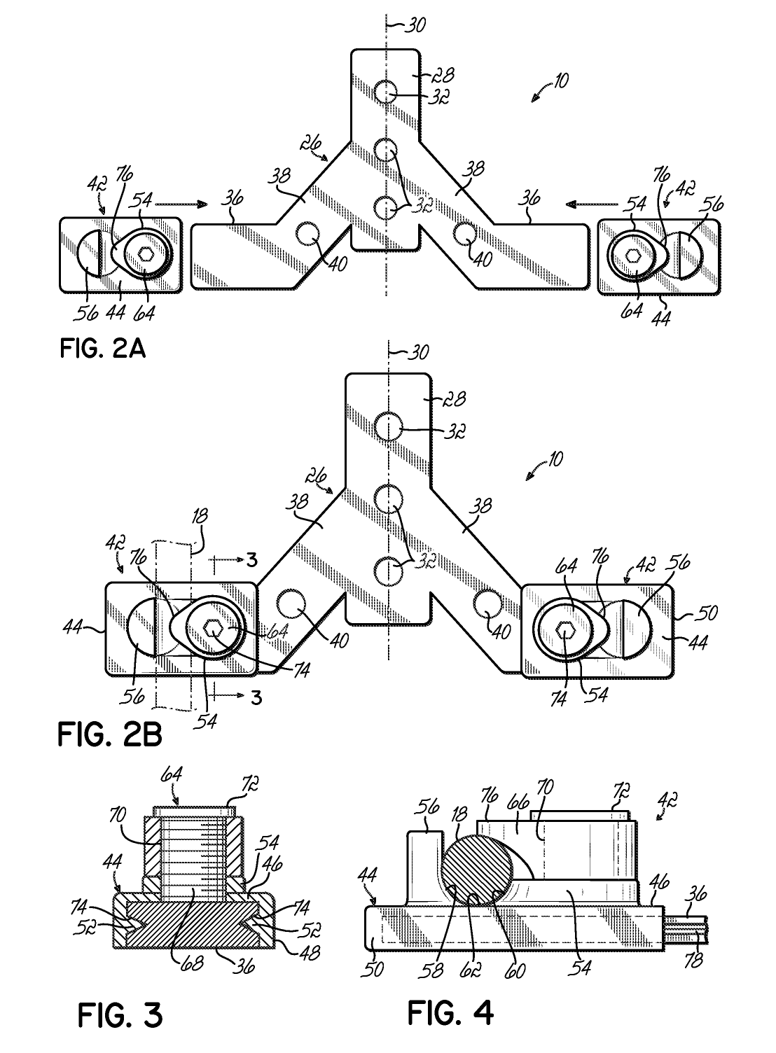

As shown generally in FIG. 1 and more specifically in FIGS. 2A-2B, the occipital plate 10 according to one embodiment of this invention includes an attachment assembly 42 mounted to each lateral section 36 of the base plate 26. The attachment assembly 42 is adjustable relative to the midline 30 of the central section 28 of the occipital plate 10 to provide for selective positioning of the rod 18 attached to the occipital plate 10 by the attachment assembly 42.

In one embodiment, each rod attachment assembly 42 includes a channel-shaped slide member 44 having an upper wall 46 and a pair of spaced sidewalls 48 projecting generally perpendicularly from the upper wall 46. A distal end of each slide member 44 includes an end cap 50 and each sidewall 48 includes a triangular-shaped extension 52 projecting inwardly toward the opposing sidewall 48. Each slide member 44 also includes a generally cylindrical boss 54 projecting upwardly from the top wall 46 of the slide member 44 opposite from the end cap 50. A post 56 extends upwardly from the top wall 46 of the slide member 44 at a location spaced between the end cap 50 and the boss 54. The post 56 has a semi-circular cross-section and includes an arcuate convex-shaped trunk portion 58 confronting an arcuate convex-shaped skirt 60 of the boss 54. The trunk 58 and skirt 60 combine to form an arcuate-shaped well 62 into which the rod 18 of the OCT construct 12 is received.

The rod 18 is clamped to the attachment assembly 42 and, as a result, the occipital plate 10 by a set screw 64 and cam lock 66 as shown specifically in FIG. 4. The set screw 64 includes a threaded shaft 68 that is received in a threaded hole 70 in the boss 54 and upper wall 46 of the slide member 44. A head 72 of the set screw 64 sits atop the cam 66. The head 72 of the set screw 64 includes hex-shaped socket 74 to receive a wrench or other driver (not shown) to adjust the set screw 64 relative to the boss 54 and slide member 44 and the lateral section 36 of the base plate 26.

A lug 76 projects from the cam lock 66 toward the post 56 and in combination with the well 62 formed by the trunk 58 and the skirt 60 secures the rod 18 to the attachment assembly 42. The distal end of the shaft 68 of the set screw 64 is juxtaposed against the upper face of the lateral section 36 of the base plate 26 as shown in FIG. 3. Advantageously, the attachment assembly according to various embodiments of this invention is securely mounted at a selected position relative to the midline of the base plate without an aperture or through hole in the base plate in that the set screw is tightened against the upper face of the lateral section.

The spaced side edges of each lateral section 36 of the base plate 26 each include a triangular-shaped groove 78 adapted to mate with one of the extensions 52 projecting from the sidewall 48 of the slide member 44 thereby forming a dove-tail joint construct between the attachment assembly 42 and the base plate 26 (FIGS. 3-4). Advantageously, rotation of the set screw 64 to secure the slide member 44 at a desired position on the lateral section 36 of the base plate 26 likewise clamps the lug 76 of the cam lock 66 onto the rod 18. As such, the surgeon is able to both secure the rod 18 relative to the midline 30 and the position of the attachment assembly 42 relative to the base plate 26 by a single operation, setting the set screw.

Therefore, the occipital plate 10 according to this invention provides for medial lateral adjustability when attaching the rods 18 as part of an OCT construct 12. The surgeon only needs to tighten one set screw 64 for each attachment assembly 42 to lock the rod 18 and the position of the attachment assembly 42 relative to the midline 30 of the occipital plate 10. Those of ordinary skill in the art will readily appreciate that other attachment assemblies and mechanisms can be utilized within the scope of this invention for securing the position of the rod relative to the midline of the occipital plate. Likewise, other geometries and configurations of the base plate are readily envisioned within the scope of this invention.

The attachment assembly may be securely and selectively mounted to the occipital plate at a variety of positions and locations on the lateral section of the base plate thereby providing for a wide range of compatibility for a variety of patient geometries, sizes and physical dimensions. Additionally, the attachment assemblies are easily and quickly secured and adjusted by the surgeon during installation of the occipital plate and OCT construct thereby minimizing surgical time and trauma to the patient. Moreover, the respective attachment assemblies can be individually positioned relative to the midline depending on the particular requirements and demands of the OCT construct and installation procedure. In other words, each of the attachment assemblies do not need to be the same distance from the midline to thereby accommodate unique geometries and installation requirements without additional inventory.

From the above disclosure of the general principles of the present invention and the preceding detailed description of at least one preferred embodiment, those skilled in the art will readily comprehend the various modifications to which this invention is susceptible. Therefore, we desire to be limited only by the scope of the following claims and equivalents thereof.

We claim:

1. A fixation system for connecting a stabilization system to a bone, the fixation system comprising:
a base plate having a midline and adapted to be mounted to the bone;
at least one attachment assembly coupled to the base plate at a variety of positions relative to the midline of the base plate;
a joint construct mounting the attachment assembly to the base plate; and
a rod selectively coupled to the base plate via the attachment assembly at a variety of positions relative to the midline of the base plate.

2. The fixation system of claim 1 further comprising a pair of the attachment assemblies coupled to the base plate on opposite sides of the midline and a pair of the rods each selectively coupled to one of the attachment assemblies.

3. The fixation system of claim 1 wherein the joint construct is a dove tail connection.

4. The fixation system of claim 1 wherein the joint construct further comprises:
an extension on one of the attachment assembly and the base plate; and
a groove on the other of the attachment assembly and the base plate.

5. The fixation system of claim 1 wherein the attachment assembly is coupled to the base plate without the benefit of any aperture in the base plate.

6. The fixation system of claim 1 wherein the base plate further comprises:
a central section aligned with the midline; and
a lateral section spaced from the midline and coupled to the attachment assembly.

7. The fixation system of claim 1 wherein the attachment assembly further comprises:
a slide member;
a set screw threadably coupled to the slide member; and
a cam mounted to the slide member by the set screw and adapted to secure the rod relative to the attachment assembly.

8. The fixation system of claim 7 wherein the joint construct further comprises:
an extension on one of the slide member and the base plate; and
a groove on the other of the slide member and the base plate.

9. The fixation system of claim 8 wherein slide member is coupled to the base plate via a dove tail connection.

10. The fixation system of claim 1 wherein the joint construct limits movement of the attachment assembly relative to the base plate to a direction perpendicular to the midline of the base plate.

11. An occipito-cervico-thoracic (OCT) construct comprising:
an occipital plate adapted to be mounted to an occipital bone;
a pair of rods releasably secured in space relation to the occipital plate;
a plurality of vertebral mounts each connecting one of the rods to selected vertebrae;
the occipital plate further comprising,
(a) a base plate having a midline and adapted to be mounted to the bone;
(b) a pair of attachment assemblies each coupled to the base plate on opposite sides of the midline at a variety of positions relative to the midline of the base plate; and
(c) a pair of joint constructs each mounting one of the attachment assemblies to the base plate.

12. The OCT construct of claim 11 wherein each attachment assembly further comprises:
a slide member;
a set screw threadably coupled to the slide member; and
a cam mounted to the slide member by the set screw and adapted to secure the associated rod relative to the attachment assembly.

13. The OCT construct of claim 12 wherein each joint construct further comprises:
an extension on one of the slide member and the base plate; and
a groove on the other of the slide member and the base plate.

14. The OCT construct of claim 11 wherein each joint construct limits movement of the associated attachment assembly relative to the base plate to a direction perpendicular to the midline of the base plate.

15. An occipital plate assembly to mount a pair of rods to an occipital bone, the occipital plate assembly comprising:
a base plate having a midline and adapted to be mounted to the occipital bone;
a pair of attachment assemblies each coupled to the base plate at a variety of positions relative to the midline of the base plate and adapted to be secured to one of the rods; and
a pair of joint constructs each mounting one of the attachment assemblies to the base plate.

16. The occipital plate assembly of claim 15 wherein each attachment assembly further comprises:
a slide member;
a set screw threadably coupled to the slide member; and
a cam mounted to the slide member by the set screw and adapted to secure the associated rod relative to the attachment assembly.

17. The occipital plate assembly of claim 16 wherein each joint construct further comprises:
an extension on one of the slide member and the base plate; and
a groove on the other of the slide member and the base plate.

18. The occipital plate assembly of claim 15 wherein each joint construct limits movement of the associated attachment assembly relative to the base plate to a direction perpendicular to the midline of the base plate.

19. The occipital plate assembly of claim 15 wherein attachment assembly is coupled to the base plate via a dove tail connection.
